(19) United States
(12) Patent Application Publication
(10) Pub. No.: US 2025/0296918 A1
(43) Pub. Date: Sep. 25, 2025

(54) METHOD FOR PRODUCING MERCAPTO HETEROCYCLIC COMPOUND AND METHOD FOR TESTING THE SAME

(71) Applicant: Resonac Corporation, Tokyo (JP)

(72) Inventors: Manabu KUWAJIMA, Minato-ku, Tokyo (JP); Keiichi NAKAMURA, Minato-ku, Tokyo (JP)

(73) Assignee: Resonac Corporation, Tokyo (JP)

(21) Appl. No.: 18/860,220

(22) PCT Filed: Apr. 25, 2023
(86) PCT No.: PCT/JP2023/016194

§ 371 Date: Oct. 25, 2024

(30) Foreign Application Priority Data
Apr. 28, 2022 (JP) 2022-075267

(51) Int. Cl. C07D 307/33; G01N 1/18; G01N 21/78

(52) U.S. Cl. CPC C07D 307/33; G01N 1/18; G01N 21/78

(57) ABSTRACT

A method for producing a purified mercapto heterocyclic compound includes: a step (1) of mixing a specimen fractionated from a mercapto heterocyclic compound with an indicator to obtain a test solution; and a step (2) of observing the presence or absence of coloration of the test solution obtained in the step (1) to determine that the mercapto heterocyclic compound having coloration contains an impurity; and carrying out a step (3) of removing an impurity from the mercapto heterocyclic compound when the mercapto heterocyclic compound is determined to contain the impurity in the step (2).

### TECHNICAL FIELD

The present invention relates to a method for producing a mercapto heterocyclic compound and a method for testing the same.

### BACKGROUND ART

Mercapto heterocyclic compounds are used as synthetic raw materials for pharmaceuticals, agrochemicals, and cosmetics, as well as active ingredients of drugs. For example, Patent Literature 1 discloses that 5-(2-hydroxyethyl)-4-thiazolidone synthesized using mercaptobutyrolactone can be used as an anticonvulsant, an antipyretic, for example, Patent Literature 2 discloses that a mercaptolactone compound having a predetermined structure can be used as an anti-inflammatory compound and an anti-allergic compound for use in the treatment of asthma and other inflammatory diseases. Patent Literature 3 discloses a mercapto heterocyclic compound useful as a synthetic raw material for medicines and agrochemicals or as a permanent agent.

### CITATION LIST

### Patent Literature

Patent Literature 1: U.S. Pat. No. 3,328,415B
Patent Literature 2: Japanese Translation of PCT International Application Publication No. 1999-501675
Patent Literature 3: JP2008-7501A

### SUMMARY OF INVENTION

### Technical Problem

As described above, when mercapto heterocyclic compounds are used as synthetic raw materials for pharmaceuticals, agrochemicals, and cosmetics, or as active ingredients of drugs, the mercapto heterocyclic compound is desirably free of, for example, impurities such as unreacted products or by-products during production and metals such as iron, chromium, and nickel derived from the production line. In the production of a mercapto heterocyclic compound, therefore, the impurities have conventionally been confirmed after the production by using a gas chromatograph, a high-performance liquid chromatography a quadrupole mass spectrometer, an inductively coupled plasma atomic emission spectrometer, an inductively coupled plasma mass spectrometer, and a combination thereof.

However, these analyzers are expensive and have problems in terms of production and quality control, for example, complicated analytical operation and time-consuming analyses.

The present invention has been made in view of the above problems, and an object thereof is to provide a method for producing a purified (high-purity) mercapto heterocyclic compound by determining the presence or absence of impurities in a mercapto heterocyclic compound using a simple method and removing any impurities, if any; and a test method for determining the presence or absence of impurities in a mercapto heterocyclic compound using a simple method.

### Solution to Problem

The present inventors have carried out intensive studies and as a result have found that the presence or absence of impurities in a mercapto heterocyclic compound can be determined by mixing the mercapto heterocyclic compound with a predetermined indicator to determine the presence or absence of coloration by the indicator, and have completed the present invention. That is, the present invention includes the following [1] to [10].

[1] A method for producing a purified mercapto heterocyclic compound, the method including: a step (1) of mixing a specimen fractionated from a mercapto heterocyclic compound with an indicator to obtain a test solution; and a step (2) of observing the presence or absence of coloration of the test solution obtained in the step (1) to determine that the mercapto heterocyclic compound having coloration contains an impurity; and
carrying out a step (3) of removing an impurity from the mercapto heterocyclic compound when the mercapto heterocyclic compound is determined to contain the impurity in the step (2).

[2] The method for producing a purified mercapto heterocyclic compound according to [1], wherein the mercapto heterocyclic compound is represented by the following formula (1):

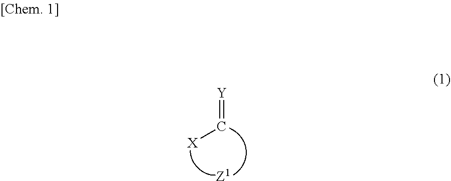

wherein X represents any of structures —O—, —S—, —NH—, and —NR¹—; R¹ represents an alkyl group, an alkoxy group, or an alkoxyalkyl group, having 1 to 6 carbon atoms; Y represents an oxygen atom, a sulfur atom, or NR²; R² represents a hydrogen atom or an alkyl group having 1 to 6 carbon atoms; and Z′ represents a divalent organic residue having at least one mercapto group.

[3] The method for producing a purified mercapto heterocyclic compound according to [1] or [2], wherein the mercapto heterocyclic compound is at least one selected from the group consisting of 2-mercapto-4-butyrolactone (also known as 2-mercapto-4-butanolide), 2-mercapto-4-methyl-4-butyrolactone, 2-mercapto-4-ethyl-4-butyrolactone, and 2-mercapto-4-butyrothiolactone.

[4] The method for producing a purified mercapto heterocyclic compound according to any one of [1] to [3], wherein the mercapto heterocyclic compound is 2-mercapto-4-butyrolactone (also known as 2-mercapto-4-butanolide).

[5] The method for producing a purified mercapto heterocyclic compound according to any one of [1] to [4], wherein the indicator is at least one selected from the group consisting of pure water, a basic compound, and a basic aqueous solution.

[6] The method for producing a purified mercapto heterocyclic compound according to any one of [1] to [5], wherein the indicator is at least one selected from the group consisting of a tetrasodium etidronate aqueous solution, a tetrasodium ethylenediaminetetraacetate aqueous solution, and an aqueous solution of alkali metal hydroxide.

[7] The method for producing a purified mercapto heterocyclic compound according to any one of [1] to [6], wherein the step (3) is a step of removing the impurity from the mercapto heterocyclic compound by distillation or active carbon.

[8] A method for testing a mercapto heterocyclic compound, the method including:
a step (1) of mixing a specimen fractionated from a mercapto heterocyclic compound with an indicator to obtain a test solution; and
a step (2) of observing the presence or absence of coloration of the test solution obtained in the step (1) to determine that the mercapto heterocyclic compound having coloration contains an impurity.

[9] The method for testing a mercapto heterocyclic compound according to [8], wherein the mercapto heterocyclic compound is 2-mercapto-4-butyrolactone (also known as 2-mercapto-4-butanolide).

[10] The method for testing a mercapto heterocyclic compound according to [8] or [9], wherein the indicator is at least one selected from the group consisting of pure water, a basic compound, and a basic aqueous solution.

### Advantageous Effects of Invention

The present invention can provide a method for producing a purified mercapto heterocyclic compound by determining the presence or absence of impurities in a mercapto heterocyclic compound using a simple method and removing any impurities, if any; and a test method for determining the presence or absence of impurities in a mercapto heterocyclic compound using a simple method.

### BRIEF DESCRIPTION OF DRAWING

FIGS. 1(1) and (2) are photographs showing the presence or absence of coloration of test solutions of 2-mercapto-4-butyrolactone mixed with each indicator.

FIG. 2(1) is a photograph showing differences in coloration upon addition of a tetrasodium etidronate aqueous solution due to differences in the concentration of iron in 2-mercapto-4-butyrolactone. FIG. 2(2) is a graph showing the relationship between the concentration of iron in 2-mercapto-4-butyrolactone and chromaticity (a*).

FIG. 3 is a photograph showing differences in coloration due to differences in the amount of a tetrasodium etidronate aqueous solution added to 2-mercapto-4-butyrolactone.

### DESCRIPTION OF EMBODIMENT

Hereinafter, suitable embodiments of the present invention will be described. The embodiments described below are given as typical examples of the embodiments of the present invention, which are not to be construed as narrowing the scope of the present invention.

### <Method for Producing Mercapto Heterocyclic Compound>

An embodiment of the present invention is a method for producing a purified mercapto heterocyclic compound, the method including: a step (1) of mixing a specimen fractionated from a mercapto heterocyclic compound with an indicator to obtain a test solution; a step (2) of observing the presence or absence of coloration of the test solution obtained in the step (1) to determine that the mercapto heterocyclic compound having coloration contains an impurity; and a step (3) of removing an impurity from the mercapto heterocyclic compound when the mercapto heterocyclic compound is determined to contain the impurity in the step (2).

### [Step (1)]

The step (1) is a step of mixing a specimen fractionated from a mercapto heterocyclic compound with an indicator to obtain a test solution.

### <Mercapto Heterocyclic Compound>

The mercapto heterocyclic compound is not particularly limited as long as it is a compound having a mercapto group and a heterocyclic structure, and preferably a mercapto heterocyclic compound represented by the following formula (1).

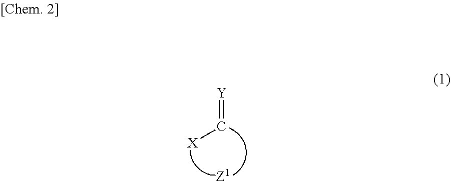

In the formula (1), X represents any of structures —O—, —S—, —NH—, and —NR¹—. R¹ represents an alkyl group having 1 to 6 carbon atoms, an alkoxy group having 1 to 6 carbon atoms, or an alkoxyalkyl group having 1 to 6 carbon atoms. Among these, R¹ is preferably an alkyl group having 1 to 4 carbon atoms, an alkoxy group having 1 to 4 carbon atoms, and an alkoxyalkyl group having 1 to 4 carbon atoms, and in particular, a methyl group, an ethyl group, a methoxy group, an ethoxy group, a methoxyethyl group, and an ethoxyethyl group are more preferable from the viewpoint of industrial availability of raw materials and handleability.

In the formula (1), Y represents an oxygen atom, a sulfur atom, or NR². R² represents a hydrogen atom or an alkyl group having 1 to 6 carbon atoms. Among these, R² is preferably a hydrogen atom, a methyl group, an ethyl group from the viewpoint of industrial availability of raw materials and handleability. Among these, Y is more preferably an oxygen atom from the viewpoint of industrial availability of raw materials and handleability.

In the formula (1), Z¹ represents a divalent organic residue having at least one mercapto group (—SH). The number of mercapto groups may be one or more, and is more preferably one. The organic residue Z¹ is preferably a hydrocarbon group to which a mercapto group is bonded and may have a branch or a side chain. Examples of the side chain include an alkyl group and an alkenyl group.

The divalent organic residue is preferably an alkylene group to which a mercapto group is bonded. The position at which the mercapto group is bonded to the alkylene group is not particularly limited. The mercapto group may be bonded directly to the alkylene group or may be bonded via another alkylene group, for example, a mercaptoethyl group bonded to a carbon atom in the alkylene group.

When the compound of the formula (1) is used as a permanent wave agent, the reactivity of the mercapto groups in the compound of the formula (1) to a cystine bond in the hair is higher. The mercapto group is preferably bonded directly to the alkylene group.

Examples of the mercapto heterocyclic compound represented by the formula (1) include 2-mercapto-3-propiolactone, 2-mercapto-2-methyl-3-propiolactone, 2-mercapto-3-methyl-3-propiolactone, 2-mercapto-3-ethyl-3-propiolactone, 2-mercapto-2,3-dimethyl-3-propiolactone, 2-mercapto-3-propiolactam, 2-mercapto-2-methyl-3-propiolactam, 2-mercapto-3-methyl-3-propiolactam, 2-mercapto-3-ethyl-3-propiolactam, 2-mercapto-2,3-dimethyl-3-propiolactam, 2-mercapto-3-propiothiolactone, 2-mercapto-2-methyl-3-propiothiolactone, 2-mercapto-3-methyl-3-propiothiolactone, 2-mercapto-3-ethyl-3-propiothiolactone, 2-mercapto-2,3-dimethyl-3-propiothiolactone, 3-mercapto-4-butyrolactone, 2,3-dimercapto-4-butyrolactone, 2,4-dimercapto-4-butyrolactone, 3,4-dimercapto-4-butyrolactone, 3-mercapto-4-butyrothiolactone, 3-mercapto-4-butyrolactam, 2,3-dimercapto-4-butyrolactam, 2,4-dimercapto-4-butyrolactam, 3,4-dimercapto-4-butyrolactam, 2-mercapto-4-butyrolactone (also known as 2-mercapto-4-butanolide), 2-mercapto-2-methyl-4,4-dimethyl-4-butyrolactone, 2-mercapto-3-(2-propenyl)-4-butyrolactone, 2-mercapto-4-methyl-4-butyrolactone, 2-mercapto-2-methyl-4-butyrolactone, 2-mercapto-3-methyl-4-butyrolactone, 2-mercapto-4-methyl-4-butyrolactone, 2-mercapto-3,4-dimethyl-4-butyrolactone, 2-mercapto-2-ethyl-4-butyrolactone, 2-mercapto-3-ethyl-4-butyrolactone, 2-mercapto-4-ethyl-4-butyrolactone, 2-mercapto-4-butyrothiolactone, 2-mercapto-2-methyl-4-butyrothiolactone, 2-mercapto-3-methyl-4-butyrothiolactone, 2-mercapto-4-methyl-4-butyrothiolactone, 2-mercapto-3,4-dimethyl-4-butyrothiolactone, 2-mercapto-2-ethyl-4-butyrothiolactone, 2-mercapto-3-ethyl-4-butyrothiolactone, 2-mercapto-4-ethyl-4-butyrothiolactone, 2-mercapto-4-butyrolactam, 2-mercapto-2-methyl-4-butyrolactam, 2-mercapto-3-methyl-4-butyrolactam, 2-mercapto-4-methyl-4-butyrolactam, 2-mercapto-3,4-dimethyl-4-butyrolactam, 2-mercapto-2-ethyl-4-butyrolactam, 2-mercapto-3-ethyl-4-butyrolactam, and 2-mercapto-4-ethyl-4-butyrolactam;
3-mercapto-5-valerolactone, 4-mercapto-5-valerolactone, 2,3-dimercapto-5-valerolactone, 2,4-dimercapto-5-valerolactone, 2,5-dimercapto-5-valerolactone, 3,4-dimercapto-5-valerolactone, 3-mercapto-5-valerothiolactone, 3-mercapto-5-valerolactam, 4-mercapto-5-valerolactam, 2,3-dimercapto-5-valerolactam, 2,4-dimercapto-5-valerolactam, 2,5-dimercapto-5-valerolactam, 2-mercapto-5-valerolactone, 2-mercapto-2-methyl-5-valerolactone, 2-mercapto-3-methyl-5-valerolactone, 2-mercapto-4-methyl-5-valerolactone, 2-mercapto-5-methyl-5-valerolactone, 2-mercapto-2-ethyl-5-valerolactone, 2-mercapto-3-ethyl-5-valerolactone, 2-mercapto-4-ethyl-5-valerolactone, 2-mercapto-5-ethyl-5-valerolactone, 2-mercapto-5-valerolactam, 2-mercapto-2-methyl-5-valerolactam, 2-mercapto-3-methyl-5-valerolactam, 2-mercapto-4-methyl-5-valerolactam, 2-mercapto-5-methyl-5-valerolactam, 2-mercapto-2-ethyl-5-valerolactam, 2-mercapto-3-ethyl-5-valerolactam, 2-mercapto-4-ethyl-5-valerolactam, 2-mercapto-5-ethyl-5-valerolactam, 2-mercapto-5-valerothiolactone, 2-mercapto-2-methyl-5-valerothiolactone, 2-mercapto-3-methyl-5-valerothiolactone, 2-mercapto-4-methyl-5-valerothiolactone, 2-mercapto-5-methyl-5-valerothiolactone, 2-mercapto-2-ethyl-5-valerothiolactone, 2-mercapto-3-ethyl-5-valerothiolactone, 2-mercapto-4-ethyl-5-valerothiolactone, and 2-mercapto-5-ethyl-5-valerothiolactone; and
3-mercapto-6-hexanolactone, 4-mercapto-6-hexanolactone, 5-mercapto-6-hexanolactone, 2,3-dimercapto-6-hexanolactone, 2,4-dimercapto-6-hexanolactone, 2,5-dimercapto-6-hexanolactone, 3-mercapto-6-hexanolactam, 4-mercapto-6-hexanolactam, 5-mercapto-6-hexanolactam, 2,3-dimercapto-6-hexanolactam, 2,4-dimercapto-6-hexanolactam, 2,5-dimercapto-6-hexanolactam, 2-mercapto-6-hexanolactone, 2-mercapto-2-methyl-6-hexanolactone, 2-mercapto-3-methyl-6-hexanolactone, 2-mercapto-4-methyl-6-hexanolactone, 2-mercapto-5-methyl-6-hexanolactone, 2-mercapto-6-methyl-6-hexanolactone, 2-mercapto-6-hexanolactam, 2-mercapto-2-methyl-6-hexanolactam, 2-mercapto-3-methyl-6-hexanolactam, 2-mercapto-4-methyl-6-hexanolactam, 2-mercapto-5-methyl-6-hexanolactam, 2-mercapto-6-methyl-6-hexanolactam, 2-mercapto-6-hexanothiolactone, 2-mercapto-2-methyl-6-hexanothiolactone, 2-mercapto-3-methyl-6-hexanothiolactone, 2-mercapto-4-methyl-6-hexanothiolactone, 2-mercapto-5-methyl-6-hexanothiolactone, 2-mercapto-6-methyl-6-hexanothiolactone, 2-mercapto-7-heptanolactone, 2-mercapto-7-heptanothiolactone, 2-mercapto-7-heptanolactam, 2-mercapto-8-octanolactone, 2-mercapto-8-octanothiolactone, 2-mercapto-8-octanolactam, 2-mercapto-9-nonalactone, 2-mercapto-9-nonathiolactone, 2-mercapto-9-nonalactam, and N-alkyl derivatives (e.g., N-methyl or N-ethyl derivatives), N-alkoxy derivatives (e.g., N-methoxy or N-ethoxy derivatives), or N-alkylalkoxy derivatives (e.g., N-(2-methoxy)ethyl or N-(2-ethoxy)ethyl derivatives) of these lactams.

Among these, from the viewpoint of the interaction between the mercapto heterocyclic compound and impurities, the mercapto heterocyclic compound is preferably at least one selected from the group consisting of 2-mercapto-4-butyrolactone, 2-mercapto-4-methyl-4-butyrolactone, 2-mercapto-4-ethyl-4-butyrolactone, and 2-mercapto-4-butyrothiolactone, and more preferably 2-mercapto-4-butyrolactone.

The mercapto heterocyclic compound can be synthesized by a known method. For example, the mercapto heterocyclic compound can be synthesized by the method described in JP5192730B, specifically, by reacting a metal sulfide such as sodium sulfide, potassium sulfide, calcium sulfide, and magnesium sulfide, or a metal hydrosulfide such as sodium hydrosulfide and potassium hydrosulfide with a predetermined compound such as 2-chloro-4-butyrolactone, 2-bromo-4-butyrolactone, 2-iodo-4-butyrolactone, 2,3-dichloro-5-valerolactam, 2,3-dibromo-5-valerolactam, and 2,3-diiodo-5-valerolactam in the presence of a solvent such as water, methanol, acetone, 1,4-dioxane, 1,2-dimethoxyethane, methyl-tert-butyl ether (MTBE), tetrahydrofuran (THE), diethyl ether, N, N-dimethylformamide (DMF), and N-methyl pyrrolidone under conditions of pH 7.0 to 11.0 and 40° C. or lower.

In step (1), the mercapto heterocyclic compound is a mercapto heterocyclic compound after the reaction of the mercapto heterocyclic compound as described above and before or after the removal of impurities. The mercapto heterocyclic compound after the reaction may be, for example, a mercapto heterocyclic compound in a reactor, a mercapto heterocyclic compound that undergoes a separation and purification process after the reaction, a mercapto heterocyclic compound extracted on the way to a storage container after purification, a mercapto heterocyclic compound after a certain period of time has passed after being transferred to the storage container following purification, or a mercapto heterocyclic compound after a certain period of time has passed, such as after being filled in a product container.

If a purified mercapto heterocyclic compound by distillation or active carbon as in the step (3) described below is used as the mercapto heterocyclic compound, the effect of removing an impurity can also be confirmed.

The specimen to be fractionated from a mercapto heterocyclic compound can be obtained by an arbitrary method. For example, the specimen may be fractionated from a reactor, a storage container, or a product container containing the mercapto heterocyclic compound using a pipette, for example, or mercapto heterocyclic compound may be directly placed in a container for analysis to obtain the specimen. The amount of the specimen is not particularly limited, but is 1 g or more, preferably 10 g or more, and more preferably 100 g or more, from the viewpoint of accurately determining the presence or absence of coloration in the step (2) described below.

### <Indicator>

An indicator to be mixed with a specimen fractionated from the mercapto heterocyclic compound is used to determine the presence or absence of impurities in the mercapto heterocyclic compound. When the specimen contains an impurity, the indicator causes a color reaction with the impurity to produce a predetermined color.

The indicator is not particularly limited as long as it is brought into contact with the impurity in the mercapto heterocyclic compound to cause coloration, but is preferably at least one selected from pure water, a basic compound, and a basic aqueous solution. Examples of the basic compound include solid potassium hydroxide, solid sodium hydroxide, and pyridine. Examples of the basic aqueous solution include an alkaline metal hydroxide aqueous solution, an alkaline earth metal aqueous solution, an etidronate aqueous solution, and an ethylenediaminetetraacetate aqueous solution. More preferably, the indicator is a basic aqueous solution, and the aqueous solution contains at least one selected from the group consisting of etidronate, ethylenediamine tetraacetate, and alkali metal hydroxide. The indicator is still more preferably a basic aqueous solution containing at least one selected from the group consisting of tetrasodium etidronate, tetrasodium ethylenediaminetetraacetate, sodium hydroxide, and potassium hydroxide, and particularly preferably a basic aqueous solution containing tetrasodium etidronate.

The addition amount of the indicator to be mixed with the specimen can be appropriately set in consideration of, for example, the amount of impurities in the mercapto heterocyclic compound and the visibility of the test solution, but is 0.01 parts by mass or more, preferably 0.1 parts by mass or more, and more preferably 1 part by mass or more based on 100 parts by mass of the mercapto heterocyclic compound in order to accurately determine the presence or absence of coloration.

### [Step (2)]

The step (2) is a step of observing the presence or absence of coloration of the test solution obtained in the step (1) to determine that the mercapto heterocyclic compound having coloration contains an impurity.

### <Impurity>

The details of impurities are unknown, but it is assumed that the impurities include metals derived from the metal container used for the synthesis, and debris and rust from iron pipes, for example, in contact with the mercapto heterocyclic compound during transportation or storage. The type of metal is assumed to be, for example, iron, chromium, or nickel.

The principle of coloration of the test solution is not well understood. When a metal is contained as an impurity, it is presumed that the metal and the mercapto group of the mercapto heterocyclic compound interact with each other. The coloration is significant when the mercapto heterocyclic compound has a lactone ring and the indicator is a basic aqueous solution, which may be because the compound produced by hydrolysis of the lactone ring by a small amount of water cooperates with components contained in the indicator to more favorably affect the coloration.

### <Method of Determining Coloration>

The presence or absence of coloration of the test solution is usually confirmed by visual observation with the naked eye but may also be quantitatively confirmed using a color difference meter such as SD 6000 (manufactured by NIPPON DENSHOKU INDUSTRIES CO., LTD.) or a handy type spectrometer. The presence or absence of the coloration is confirmed by placing the control specimen (Blank), in which no indicator is added to the specimen fractionated from the mercapto heterocyclic compound, and the test solution with the indicator added in a sealable container such as a colorless transparent glass or plastic, stirring, and allowing to stand for 10 to 20 minutes at a humidity of 20 to 70 RH % and an ambient temperature (1 to 35° C.) under the atmospheric pressure, followed by comparison of the color tone of the solution between the control specimen (Blank) and the test solution. If, as a result of the comparison, coloration is observed due to the addition of the indicator compared to the control specimen, it is determined that the mercapto heterocyclic compound contains an impurity. The coloration means color development or discoloration of the test solution, including a change in the intensity of the color, although the color tone itself is the same.

In the above determination, for example, a color sample, such as a standard tone liquid or a standard tone table, which indicates the correlation between the intensity of coloration by each indicator and the concentration of the impurity, may be prepared in advance to compare the color sample with the test solution for the determination. It is preferable to confirm the lower limit concentration of the impurity at which the presence or absence of coloration by visual observation can be judged from the correlation between the intensity of coloration by each indicator and the concentration of the impurity.

The intensity of coloration may be determined by visual observation or may be indicated by numerical values in a color system such as CIEL *a*b* color space, RGB color space, or CMYK color space, for example, using a spectroscopic analyzer such as an ultraviolet-visible spectrophotometer and a spectrophotometer colorimeter. The concentration of the impurity can be quantified using, for example, a gas chromatograph, a high-performance liquid chromatograph, a quadrupole mass spectrometer, an inductively coupled plasma atomic emission spectrometer, an inductively coupled plasma mass spectrometer, and a combination thereof.

### [Step (3)]

The step (3) is a step of removing an impurity from the mercapto heterocyclic compound when the mercapto heterocyclic compound is determined to contain the impurity in the step (2). The method of removing the impurity is not particularly limited, but removal by distillation or active carbon is preferred, A purified mercapto heterocyclic compound can be obtained by this step. The presence or absence of impurities may be confirmed again by using the purified mercapto heterocyclic compound as the mercapto heterocyclic compound in the step (2).

### <Method for Testing Mercapto Heterocyclic Compound>

An embodiment of the present invention is a method for testing a mercapto heterocyclic compound, the method including: a step (1) of mixing a specimen fractionated from a mercapto heterocyclic compound with an indicator to obtain a test solution; and a step (2) of observing the presence or absence of coloration of the test solution obtained in the step (1) to determine that the mercapto heterocyclic compound having coloration contains an impurity.

The step (1) and the step (2) are synonymous with the content described in the section of “Method for Producing Mercapto Heterocyclic Compound” above.

### EXAMPLES

Hereinafter, the present invention will be specifically described based on Examples, but the present invention is not limited to the Examples and can be appropriately modified as long as the gist thereof is not changed, to be practiced.

### [Measurement Methods]

In the present invention, each measurement method is as follows.

### <Content of Metal>

The content of iron in 2-mercapto-4-butyrolactone was measured by the following procedure.

Preparation of analytical sample: 0.5 mL of 2-mercapto-4-butyrolactone was weighed in a quartz beaker, 3 mL of concentrated sulfuric acid was added thereto, and the mixture was heated to 250° C. on a hot plate for carbonization. Further, 1 mL of concentrated nitric acid was added and heated again to 250° C. When the generation of brown smoke subsided, the mixture was taken off the hot plate and cooled. The addition of concentrated nitric acid and heating were repeated until brown smoke was no longer generated while a degradation liquid thereof became colorless or pale yellow. The degradation liquid was transferred to a 50-mL measuring flask, and the flask was filled up to the marked line with water. Thus, an analytical sample was obtained.

Analysis of analytical sample: The prepared analytical sample was measured with an inductively coupled plasma atomic emission spectrometer as described below.

Instrument used: Inductively coupled plasma atomic emission spectrometer (product name: 700 Series ICP-OES (manufactured by Agilent Technologies)

### <Spectroscopic Analysis>

The spectroscopic analysis of the test solution in which 2-mercapto-4-butyrolactone and the indicator were mixed was performed under the following conditions to obtain chromaticity (a*).

Instrument used: SD 6000 (manufactured by NIPPON DENSHOKU INDUSTRIES CO., LTD.)

Measurement condition: 20 g of the sample was placed in a 5 cm cell and analyzed by a transmission method.

### [Synthesis of Raw Material 1]

2-Mercapto-4-butyrolactone was produced by the following method. In a SUS container, 70% sodium hydrosulfide (manufactured by Junsei Chemical Co., Ltd.) was dissolved in a mixed solvent of 69.4 parts by mass of 1,2-dimethoxyethane (manufactured by Junsei Chemical Co., Ltd., special grade) and 69.4 parts by mass of purified water based on 100 parts by mass of the sodium hydrosulfide at room temperature. While the solution was stirred under ice-cooling and ordinary pressure conditions (10° C. or lower, about 0.10 MPa), hydrochloric acid (manufactured by Junsei Chemical Co., Ltd., special grade of 35% to 37%) was added thereto to adjust the pH to 8.9. While the solution was cooled to maintain the temperature at 10° C. or lower, 69.4 parts by mass of 2-bromo-4-butyrolactone (manufactured by Tokyo Chemical Industry Co., Ltd.) was added dropwise to 100 parts by mass of the sodium hydrosulfide over about 20 minutes. The reaction liquid after completion of the dropwise addition was stirred for 2 minutes.

Thereafter, hydrochloric acid was added to adjust the pH of the solution to 4.0 while cooling the solution to a temperature of 10° C. or lower. After an inorganic salt precipitated in the solution was removed by suction filtration, ethyl acetate (manufactured by Junsei Chemical Co., Ltd., special grade) was added to the filtrate side to extract the organic phase. The resulting aqueous phase was extracted again with ethyl acetate. These extracted organic phases were combined and concentrated under reduced pressure.

The concentrated solution was stored in an iron container for one day, followed by fractionation of 2-mercapto-4-butyrolactone (raw material 1). The content of iron in the fractionated 2-mercapto-4-butyrolactone was 0.5 ppm by mass, and no other metals were detected.

### [Coloration Confirmation Experiment 1]

### Examples 1 to 6

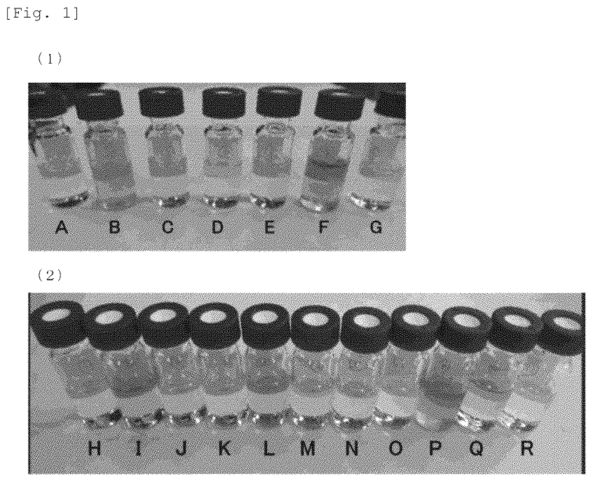

One gram of the raw material 1,2-mercapto-4-butyrolactone, was placed in each glass vial labeled A to Q. Test solutions were prepared by adding and mixing 50 mg of the following indicators to each glass vial: F: tetrasodium etidronate; B: 10% by mass of tetrasodium ethylenediaminetetraacetate aqueous solution; I: pure water; P: 8% by mass of sodium hydroxide aqueous solution; Q: potassium hydroxide (solid); and L: pyridine. The presence or absence of coloration of each test solution was observed visually with the naked eye. The results are shown in Table 1, FIG. 1(1), and FIG. 1(2).

### Comparative Examples 1 to 11

The presence or absence of coloration of each test solution was observed in the same manner as in Example 1, except that A: 85% by mass of phosphoric acid, C: 10% by mass of citric acid, D: 5% by mass of hydrochloric acid, E: acetic acid (purity: 99.7%), J: methanol, K: tetrahydrofuran, M: toluene, N: acetonitrile, and O: ethyl acetate were used as indicators of Comparative Examples instead of the indicators used in Example 1. The results are shown in Table 1, FIG. 1(1), and FIG. 1(2). G and H were blanks to which no indicator was added. Results of Examples 1 to 6 and Comparative Examples 1 to 11 are summarized in Table 1.

TABLE 1

| | Vial | | Attribute | Appearance |
|---|---|---|---|---|
| | code | Indicator | of indicator | of color |
| Comparative | A | phosphoric acid | acidic | colorless |
| Example 1 | | | | |
| Example 1 | B | EDTA-Na aq | basic | pink |
| Comparative | C | citric acid | acidic | colorless |
| Example 2 | | | | |
| Comparative | D | hydrochloric acid | acidic | colorless |
| Example 3 | | | | |
| Comparative | E | acetic acid | acidic | colorless |
| Example 4 | | | | |
| Example 2 | F | ETDR-4Na aq | basic | red |
| Comparative | G | blank | — | colorless |
| Example 5 | | | | |
| Comparative | H | blank | — | colorless |
| Example 6 | | | | |
| Example 3 | I | pure water | neutral | pink |
| Comparative | J | methanol | neutral | colorless |
| Example 7 | | | | |
| Comparative | K | THF | neutral | colorless |
| Example 8 | | | | |
| Example 4 | L | pyridine | basic | pink |
| Comparative | M | toluene | neutral | colorless |
| Example 9 | | | | |
| Comparative | N | acetonitrile | neutral | colorless |
| Example 10 | | | | |
| Comparative | O | ethyl acetate | neutral | colorless |
| Example 11 | | | | |
| Example 5 | P | NaOH aq | basic | deep red |
| Example 6 | Q | KOH solid | basic | pink |

EDTA-Na aq: Tetrasodium ethylenediaminetetraacetate aqueous solution
ETDR-4Na aq: Tetrasodium etidronate aqueous solution

### [Synthesis of Raw Material 2]

The raw material 1,2-mercapto-4-butyrolactone, was subjected to a distillation treatment to obtain 2-mercapto-4-butyrolactone (raw material 2). As a result of the analysis, no metal was detected in the raw material 2.

### [Coloration Confirmation Experiment 2]

When 50 mg of tetrasodium etidronate aqueous solution was added to 1 g of the raw material 2,2-mercapto-4-butyrolactone, no coloration was observed. Then, 1 g of another raw material 2,2-mercapto-4-butyrolactone, was placed in a glass vial labeled R, and 50 mg of R: potassium hydroxide (solid) was added to the glass vial as an indicator and mixed to prepare a test solution. When the presence or absence of coloration of the test solution was visually observed with the naked eye, no coloration was observed, and the test solution remained colorless and transparent. In the raw material 2 substantially free of iron, neither the tetrasodium etidronate aqueous solution nor potassium hydroxide was colored, which indicates that the color reaction was not caused by the reaction between 2-mercapto-4-butyrolactone itself and the indicator.

In FIGS. 1(1) and (2), the relationship between each symbol and indicator was as follows:

A: phosphoric acid; B: tetrasodium ethylenediaminetetraacetate aqueous solution; C: citric acid; D: hydrochloric acid; E: acetic acid; F: tetrasodium etidronate aqueous solution; G; Blank (not added); H: Blank (not added); I: pure water, J: methanol, K: tetrahydrofuran; L: pyridine; M: toluene; N: acetonitrile; O: ethyl acetate; P: sodium hydroxide aqueous solution; Q: potassium hydroxide (solid); and R: potassium hydroxide (solid).

FIGS. 1(1) and (2) show that since a light purple to pink coloration was observed when the basic compounds used in Coloration Confirmation Experiment 1, the tetrasodium etidronate aqueous solution (F in FIG. 1), the tetrasodium ethylenediaminetetraacetate aqueous solution (B in FIG. 1), sodium hydroxide (P in FIG. 1), potassium hydroxide (Q), and pyridine (L), were used as indicators, these basic compounds could be used as indicators for the impurities in 2-mercapto-4-butyrolactone. On the other hand, no coloration was observed when the acidic compounds, phosphoric acid (A in FIG. 1), citric acid (C in FIG. 1), hydrochloric acid (D in FIG. 1), acetic acid (E in FIG. 1), methanol (J in FIG. 1), tetrahydrofuran (K in FIG. 1), toluene (M in FIG. 1), acetonitrile (N in FIG. 1), and ethyl acetate (0) were used, indicating that they are not suitable as indicators of the present invention.

### [Coloration Confirmation Experiment 3]

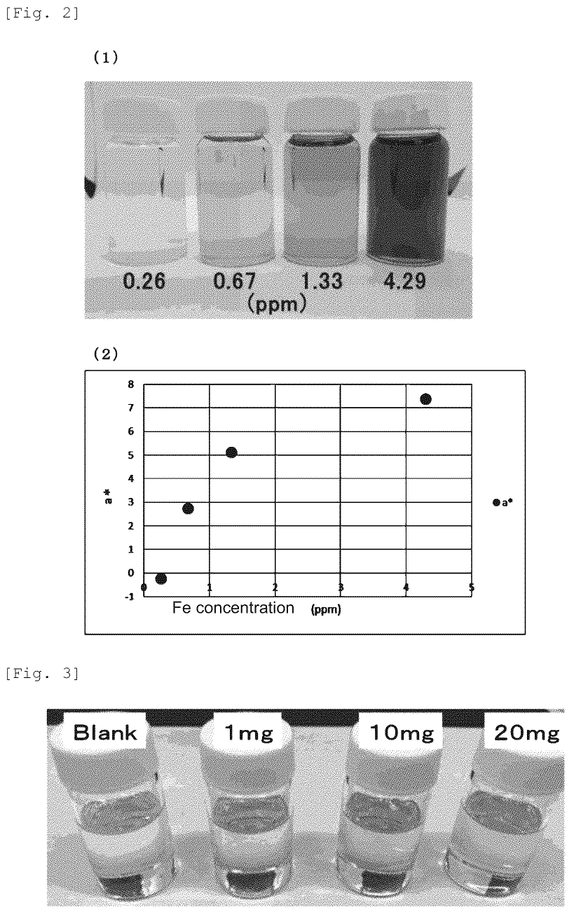

2-Mercapto-4-butyrolactone (raw material 3) was produced in the same manner as in Synthesis of Raw Material 1. The content of iron in the obtained 2-mercapto-4-butyrolactone was 0.26 ppm, 10 g of the 2-mercapto-4-butyrolactone was placed in four glass vials. Different amounts of iron powder (10 mg to 100 mg) were added to three glass vials and stirred well. 50 mg of a tetrasodium etidronate aqueous solution was each added to the four glass vials and mixed to prepare test solutions. The presence or absence of coloration of each test solution was observed. The result is shown in FIG. 2(1). The chromaticity (a*) of the test solution in each vial was measured, and the concentration of iron in the test solution in each vial was further measured. The results are summarized in FIG. 2(2).

Since the solution turned red when the chromaticity (a*) was 0 or more, from the results of FIGS. 2(1) and (2), it was shown that when the content of iron was 0.4 ppm or more, the coloration could be visually determined because the chromaticity (a*) was 0 or more.

### [Coloration Confirmation Experiment 4]

2-Mercapto-4-butyrolactone (raw material 4) was produced in the same manner as in Synthesis of Raw Material 1. The content of iron in the 2-mercapto-4-butyrolactone obtained before purification was 0.4 ppm. 10 g of the 2-mercapto-4-butyrolactone was placed in four glass vials. To each glass vial, 1 mg, 10 mg, and 20 mg of tetrasodium etidronate aqueous solution were added to measure the chromaticity (a*) of the test solution and to confirm the presence or absence of coloration. The remaining one glass vial was blank. The result is shown in FIG. 3.

The chromaticity (a*) was about 1 when 1 mg of the tetrasodium etidronate aqueous solution was added, and a light purple color was visually observed. This indicates that the presence or absence of coloration can be confirmed by adding 0.01 parts by mass of tetrasodium etidronate aqueous solution to 100 parts by mass of 2-mercapto-4-butyrolactone, and the presence or absence of impurities can be determined.

1. A method for producing a purified mercapto heterocyclic compound, the method comprising:
a step (1) of mixing a specimen fractionated from a mercapto heterocyclic compound with an indicator to obtain a test solution; and
a step (2) of observing the presence or absence of coloration of the test solution obtained in the step (1) to determine that the mercapto heterocyclic compound having coloration contains an impurity; and
carrying out a step (3) of removing an impurity from the mercapto heterocyclic compound when the mercapto heterocyclic compound is determined to contain the impurity in the step (2).

2. The method for producing a purified mercapto heterocyclic compound according to claim 1, wherein the mercapto heterocyclic compound is represented by the following formula (1): 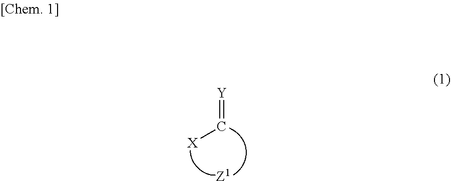
wherein X represents any of structures —O—, —S—, —NH—, and —NR¹—; R¹ represents an alkyl group, an alkoxy group, or an alkoxyalkyl group, having 1 to 6 carbon atoms; Y represents an oxygen atom, a sulfur atom, or NR²; R² represents a hydrogen atom or an alkyl group having 1 to 6 carbon atoms; and Z¹ represents a divalent organic residue having at least one mercapto group.

3. The method for producing a purified mercapto heterocyclic compound according to claim 1, wherein the mercapto heterocyclic compound is at least one selected from the group consisting of 2-mercapto-4-butyrolactone (also known as 2-mercapto-4-butanolide), 2-mercapto-4-methyl-4-butyrolactone, 2-mercapto-4-ethyl-4-butyrolactone, and 2-mercapto-4-butyrothiolactone.

4. The method for producing a purified mercapto heterocyclic compound according to claim 1, wherein the mercapto heterocyclic compound is 2-mercapto-4-butyrolactone (also known as 2-mercapto-4-butanolide).

5. The method for producing a purified mercapto heterocyclic compound according to claim 1, wherein the indicator is at least one selected from the group consisting of pure water, a basic compound, and a basic aqueous solution.

6. The method for producing a purified mercapto heterocyclic compound according to claim 1, wherein the indicator is at least one selected from the group consisting of a tetrasodium etidronate aqueous solution, a tetrasodium ethylenediaminetetraacetate aqueous solution, and an aqueous solution of alkali metal hydroxide.

7. The method for producing a purified mercapto heterocyclic compound according to claim 1, wherein the step (3) is a step of removing the impurity from the mercapto heterocyclic compound by distillation or active carbon.

8. A method for testing a mercapto heterocyclic compound, the method comprising:
a step (1) of mixing a specimen fractionated from a mercapto heterocyclic compound with an indicator to obtain a test solution; and
a step (2) of observing the presence or absence of coloration of the test solution obtained in the step (1) to determine that the mercapto heterocyclic compound having coloration contains an impurity.

9. The method for testing a mercapto heterocyclic compound according to claim 8, wherein the mercapto heterocyclic compound is 2-mercapto-4-butyrolactone (also known as 2-mercapto-4-butanolide).

10. The method for testing a mercapto heterocyclic compound according to claim 8, wherein the indicator is at least one selected from the group consisting of pure water, a basic compound, and a basic aqueous solution.

11. The method for producing a purified mercapto heterocyclic compound according to claim 2, wherein the mercapto heterocyclic compound is at least one selected from the group consisting of 2-mercapto-4-butyrolactone (also known as 2-mercapto-4-butanolide), 2-mercapto-4-methyl-4-butyrolactone, 2-mercapto-4-ethyl-4-butyrolactone, and 2-mercapto-4-butyrothiolactone.

12. The method for producing a purified mercapto heterocyclic compound according to claim 2, wherein the mercapto heterocyclic compound is 2-mercapto-4-butyrolactone (also known as 2-mercapto-4-butanolide).

13. The method for producing a purified mercapto heterocyclic compound according to claim 2, wherein the indicator is at least one selected from the group consisting of pure water, a basic compound, and a basic aqueous solution.

14. The method for producing a purified mercapto heterocyclic compound according to claim 2, wherein the indicator is at least one selected from the group consisting of a tetrasodium etidronate aqueous solution, a tetrasodium ethylenediaminetetraacetate aqueous solution, and an aqueous solution of alkali metal hydroxide.

15. The method for producing a purified mercapto heterocyclic compound according to claim 2, wherein the step (3) is a step of removing the impurity from the mercapto heterocyclic compound by distillation or active carbon.

16. The method for testing a mercapto heterocyclic compound according to claim 9, wherein the indicator is at least one selected from the group consisting of pure water, a basic compound, and a basic aqueous solution.
